(19) United States
(12) Patent Application Publication
(10) Pub. No.: US 2023/0383332 A1
(43) Pub. Date: Nov. 30, 2023

(54) METHODS AND HYDROGEL COMPOSITIONS FOR PARTITIONING BIOLOGICAL SAMPLES

(71) Applicant: Bio-Rad Laboratories, Inc., Hercules, CA (US)

(72) Inventors: Ronald LEBOFSKY, Berkeley, CA (US); Josh SHINOFF, Emeryville, CA (US); Adam M. McCOY, Davis, CA (US); Man CHENG, Danville, CA (US)

(21) Appl. No.: 18/203,424

(22) Filed: May 30, 2023

Related U.S. Application Data
(60) Provisional application No. 63/347,419, filed on May 31, 2022.

(51) Int. Cl. C12Q 1/6806; C12Q 1/6869

(52) U.S. Cl. CPC C12Q 1/6806; C12Q 1/6869; C12N 1/06

(57) ABSTRACT

Methods of making and forming partitions in nanovials are provided.

### CROSS-REFERENCE TO RELATED PATENT APPLICATIONS

The present application claims benefit of priority to U.S. Provisional Patent Application No. 63/347,419, filed May 31, 2022, which is incorporated by reference for all purposes.

### BACKGROUND OF THE INVENTION

Partitioning of reagents have been found to be useful in many molecular reactions. For example, droplet-based single cell sequencing allows for high resolution genetic sequencing.

In single cell next-generation sequencing (NGS) sample preparation, complex microfluidics are required to deliver cells and beads into barcoding partitions. This complexity increases the cost of the solution as they often require microfluidic chips as well as microfluidic controllers. Where the partitions are droplets, the chips generate droplets and the instruments perform fluidic pressure control to deliver said partitions.

Moreover, as creating the partitions is often performed as a serial operation, there is an upper limit to the number of droplets being generated. This is due to the proportional increase of time required to generate the increased number of partitions. Also, the extra time required means that cells may sit in the buffer upstream of partition generation such that the biology may start to be impacted, which will influence biological data and results.

Once the barcoding partitions are formed, there may be a need to image the contents of the partitions to analyze, for example, secreted products from the cell and then sort the cells of interest based on the imaging results. This is difficult with water in oil droplets as they are incompatible with cell sorters and microwells, which are physically fixed in space.

Lastly, canonical barcoding partitions may not allow for buffer exchange as they are fluidically isolated from each other. This prevents multistep biochemistry reactions, some of which are needed for barcoding complex cellular substrates such as methylated DNA.

### BRIEF SUMMARY OF THE INVENTION

In some embodiments, a method of linking oligonucleotides to cell nucleic acids in hollow nanovials or microwells is provided. In some embodiments, the method comprises:

loading cells into openings in hollow nanovials or microwells;
inserting one or more hydrogel bead into the openings, thereby occluding the openings and preventing diffusion of the cells from the openings;
lysing the cells in the hollow nanovials or microwells;
linking one or more oligonucleotides to a nucleic acid from the lysed cells thereby barcoding the nucleic acid, wherein the oligonucleotides are present in the hollow nanovials or microwells before the loading, or are introduced into the hollow nanovials or microwells by the hydrogel beads.

In some embodiments, the occluding comprises inducing swelling of the one or more hydrogel bead inserted into the nanovials or microwells. In some embodiments, the occluding comprises inducing swelling of the nanovials.

In some embodiments, a majority of the nanovials or microwells contain one or zero cells. In some embodiments to achieve one or zero cell loading, the lambda or, alternatively, the average number of cells as a fraction of nanovials or microwells, is approximately 0.05 to 0.1 cells per nanovial or microwell. In some embodiments to achieve one or zero cell loading, the lambda, or alternatively, the average number of cells as a fraction of nanovials or microwells is approximately 0.01 to 0.4 cells per nanovial or microwell.

In some embodiments, the oligonucleotides are linked to the hydrogel beads and are optionally released from the hydrogel beads after the sealing. In some embodiments, the oligonucleotides are linked to the hydrogel beads and are optionally released from the hydrogel beads before the linking. In some embodiments, the oligonucleotides have a barcode sequence, wherein individual hydrogel beads comprise clonal copies of the oligonucleotides and wherein the barcode sequence linked to individual hydrogel beads are unique such that the barcode distinguishes the hydrogel bead from other hydrogel beads.

In some embodiments, the oligonucleotides have a barcode sequence, wherein individual nanovials or microwells comprise clonal copies of the oligonucleotides and wherein the barcode sequenced linked to individual nanovials or microwells are unique such that the barcode distinguishes the nanovial or microwell from other nanovials or microwells.

In some embodiments, the method further comprises extracting the barcoded nucleic acids from the nanovials or microwells and combining barcoded nucleic acids from different nanovials or microwells into a bulk solution and performing nucleotide sequencing of the barcoded nucleic acids from the bulk solution.

In some embodiments, the lysing comprises diffusing one or more lysis reagents to the cells in the nanovials or microwells. In some embodiments, lysis reagents are removed from the nanovials or microwells or are inactivated before the linking.

In some embodiments, the lysing comprises diffusing protein denaturing and or degradation reagents to the cells in the nanovials or microwells. In some embodiments, lysis reagents are removed from the nanovials or microwells or are inactivated before the linking.

In some embodiments, the linking comprises annealing the oligonucleotides to the nucleic acids and optionally performing template-dependent extension of the oligonucleotide using the nucleic acid as the template.

In some embodiments, the linking comprises diffusing one or more linking reagents into the sealed nanovials or microwells containing the lysed cells. In some embodiments, the nucleic acids are DNA and the linking reagents comprise a DNA polymerase or ligase. In some embodiments, the nucleic acids are RNA and the linking reagents comprise a reverse transcriptase.

In some embodiments, the linking comprises linking the oligonucleotides to the nucleic acids via a click chemistry reaction.

In some embodiments, the hollow nanovials comprise dextran and polyethylene glycol (PEG).

In some embodiments, the hydrogel beads comprise polyacrylamide, polystyrene, silica, polymethylmethacrylate, alginate, polyethylene glycol (PEG), nylon, or agarose.

In some embodiments, the hydrogel beads comprise polyacrylamide that is cross-linked with Bis-acrylylcystamine. In some embodiments, swelling is induced in the hydrogel beads by contacting the hydrogel beads with a disulfide bond reducing agent.

In some embodiments, the hydrogel beads comprise polyacrylamide formed from polymerizing acrylamide and a photocleavable cross-linker. In some embodiments, photocleavable cross-linker is ortho-nitrobenzyl (o-NB) bis-acrylate. In some embodiments, swelling is induced in the hydrogel beads by contacting photo-cleaving the photocleavable cross-linker.

In some embodiments, a plurality of hollow nanovials or microwells comprising occluded openings that prevents diffusion of cells and released cellular products is provided. In some embodiments, at least some of the hollow nanovials or microwells comprise one or more cell and the openings are occluded by hydrogel beads blocking the openings. In some embodiments, the occluded openings are formed by introducing one or more hydrogel bead into the opening and then inducing swelling of the hydrogel bead such that the hydrogel bead plugs the opening. In some embodiments, the hollow nanovials comprise dextran and polyethylene glycol (PEG). In some embodiments, the hydrogel beads comprise polyacrylamide, polystyrene, silica, polymethylmethacrylate, alginate, polyethylene glycol (PEG), nylon, or agarose. In some embodiments, a majority of the plurality of hollow nanovials or microwells contain one or zero cells. In some embodiments, individual hydrogel beads comprise clonal copies of oligonucleotides and the oligonucleotides have a barcode sequence, and wherein the barcode sequence linked to individual hydrogel beads are unique such that the barcode distinguishes the hydrogel bead from other hydrogel beads. In some embodiments, the barcode sequences linked to individual hydrogel beads via click chemistry. In some embodiments, the oligonucleotides have a barcode sequence, wherein individual nanovials or microwells comprise clonal copies of oligonucleotides and the oligonucleotides have a barcode sequence, and wherein the barcode sequenced linked to individual nanovials or microwells are unique such that the barcode distinguishes the nanovial or microwell from other nanovials or microwells.

In some embodiments, the oligonucleotides have a barcode sequence, wherein individual nanovials comprise clonal copies of oligonucleotides and the oligonucleotides have a barcode sequence, and wherein the barcode sequences linked to individual nanovials are unique such that the barcode distinguishes the nanovial from other nanovials. In some embodiments, the barcode sequences linked to individual nanovials via click chemistry.

In some embodiments, the hydrogel beads comprise polyacrylamide that is cross-linked with Bis-acrylylcystamine.

In some embodiments, the hydrogel beads comprise polyacrylamide formed from polymerizing acrylamide and a photocleavable cross-linker. In some embodiments, the photocleavable cross-linker is ortho-nitrobenzyl (o-NB) bis-acrylate.

Also provided is a method of detecting cell proteins in hollow nanovials or microwells.

In some embodiments, the method comprises:

loading cells into openings in hollow nanovials or microwells;
inserting one or more hydrogel bead into the openings, thereby occluding the openings and preventing diffusion of the cells from the openings; and
detecting secreted cell proteins in the occluded nanovials.

In some embodiments, the nanovials or microwells comprise a binding agent (which can be but is not limited to an antibody, biotin or streptavidin) that specifically binds to one or more secreted cell protein and the secreted cell proteins bind to the binding agent, and a second binding agent (which can be directly or indirectly labeled) specific for the secreted cell protein is diffused into the occluded nanovials or microwells to specifically detect the secreted cell protein bound in the nanovials or microwells. For example, the second binding agent (e.g., an antibody or other binding agent) is typically conjugated or otherwise associated with a detectable label. The association can be direct e.g., a covalent bond, or indirect, e.g., using a secondary binding agent, chelator, or linker.

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a bead” includes a plurality of such beads and reference to “the sequence” includes reference to one or more sequences known to those skilled in the art, and so forth.

The term “amplification reaction” refers to any in vitro means for multiplying the copies of a target sequence of nucleic acid in a linear or exponential manner. Such methods include but are not limited to two-primer methods such as polymerase chain reaction (PCR); ligase methods such as DNA ligase chain reaction (see U.S. Pat. Nos. 4,683,195 and 4,683,202; PCR Protocols: A Guide to Methods and Applications (Innis et al., eds, 1990)) (LCR); QBeta RNA replicase and RNA transcription-based amplification reactions (e.g., amplification that involves T7, T3, or SP6 primed RNA polymerization), such as the transcription amplification system (TAS), nucleic acid sequence based amplification (NASBA), and self-sustained sequence replication (3 SR); isothermal amplification reactions (e.g., single-primer isothermal amplification (SPIA)); as well as others known to those of skill in the art.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing or linear amplification. In an exemplary embodiment, amplifying refers to PCR amplification using a first and a second amplification primer.

A “primer” refers to a polynucleotide sequence that hybridizes to a sequence on a target nucleic acid and serves as a point of initiation of nucleic acid synthesis. Primers can be of a variety of lengths and are often less than 50 nucleotides in length, for example 12-30 nucleotides, in length. The length and sequences of primers for use in PCR can be designed based on principles known to those of skill in the art, see, e.g., Innis et al., supra. Primers can be DNA, RNA, or a chimera of DNA and RNA portions. In some cases, primers can include one or more modified or non-natural nucleotide bases. In some cases, primers are labeled.

A nucleic acid, or a portion thereof, “hybridizes” to another nucleic acid under conditions such that non-specific hybridization is minimal at a defined temperature in a physiological buffer (e.g., pH 6-9, 25-150 mM chloride salt). In some cases, a nucleic acid, or portion thereof, hybridizes to a conserved sequence shared among a group of target nucleic acids. In some cases, a primer, or portion thereof, can hybridize to a primer binding site if there are at least about 6, 8, 10, 12, 14, 16, or 18 contiguous complementary nucleotides, including “universal” nucleotides that are complementary to more than one nucleotide partner. Alternatively, a primer, or portion thereof, can hybridize to a primer binding site if there are 0, or fewer than 2 or 3 complementarity mismatches over at least about 12, 14, 16, 18, or 20 contiguous nucleotides. In some embodiments, the defined temperature at which specific hybridization occurs is room temperature. In some embodiments, the defined temperature at which specific hybridization occurs is higher than room temperature. In some embodiments, the defined temperature at which specific hybridization occurs is at least about 37, 40, 42, 45, 50, 55, 65, 70, 75, or 80° C. In some embodiments, the defined temperature at which specific hybridization occurs is 37, 40, 42, 45, 50, 55, 60, 65, 70, 75, or 80° C.

A “template” refers to a polynucleotide sequence that comprises the polynucleotide to be amplified, adjacent to a primer hybridization site, or flanked by a pair of primer hybridization sites. Thus, a “target template” comprises the target polynucleotide sequence adjacent to at least one hybridization site for a primer. In some cases, a “target template” comprises the target polynucleotide sequence flanked by a hybridization site for a “forward” primer and a “reverse” primer.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications including but not limited to capping with a fluorophore (e.g., quantum dot) or another moiety.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.).

Polymerases include both DNA-dependent polymerases and RNA-dependent polymerases such as reverse transcriptase. At least five families of DNA-dependent DNA polymerases are known, although most fall into families A, B and C. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

As used herein, the term “partitioning” or “partitioned” refers to separating a sample into a plurality of portions, or “partitions.” Partitions are generally physical, such that a sample in one partition does not, or does not substantially, mix with a sample in an adjacent partition. Partitions can be solid or fluid. In some embodiments, a partition is a solid partition, e.g., a microchannel or microwell, or as described herein, a bead. In some embodiments, a partition is a fluid partition, e.g., a droplet. In some embodiments, a fluid partition (e.g., a droplet) is a mixture of immiscible fluids (e.g., water and oil). In some embodiments, a fluid partition (e.g., a droplet) is an aqueous droplet that is surrounded by an immiscible carrier fluid (e.g., oil). Exemplary array of wells and well descriptions can be found for example in U.S. Pat. Nos. 9,103,754 and 10,391,493. The array of wells (set of nanowells, microwells, wells) can function to capture the solid supports, optionally in addressable, known locations. As such, the array of wells can be configured to facilitate bead capture in at least one of a single-solid support format or optionally in small groups of solid supports. Exemplary microwell arrays and methods of delivery of beads to the microwells and analysis thereof is described in, e.g., PCT/US2021/034152.

As used herein a “barcode” is a short nucleotide sequence (e.g., at least about 4, 6, 8, 12, 15, 20, 50 or 75 or 100 nucleotides long or more) that identifies a molecule to which it is conjugated or from the partition or sample in which it originated. Barcodes can be used, e.g., to identify molecules originating in a partition or from a bead as later sequenced from a bulk reaction. Such a barcode can be unique for that partition or bead as compared to barcodes present in other partitions or beads, for example. For example, partitions (which can be hollow nanovials) containing target RNA from single-cells can be subject to reverse transcription conditions using primers that contain different partition-specific barcode sequence in each partition, thus incorporating a copy of a unique “cellular barcode” (because different cells are in different partitions and each partition has unique partition-specific barcodes) into the reverse transcribed nucleic acids of each partition. Thus, nucleic acid from each cell can be distinguished from nucleic acid of other cells due to the unique “cellular barcode.” In some embodiments described herein, others barcodes uniquely identify the molecule to which it is conjugated, i.e., the barcode acts as a unique molecular identifier (UMI) or yet other barcodes can indicate a sample of origin (“sample barcode”). The length of the underlying barcode sequence determines how many unique samples can be differentiated. For example, a 1 nucleotide barcode can differentiate 4, or fewer depending on degeneracy, different partitions; a 4 nucleotide barcode can differentiate 4⁴ or 256 partitions or less; a 6 nucleotide barcode can differentiate 4096 different partitions or less; and an 8 nucleotide barcode can index 65,536 different partitions or less.

An “oligonucleotide” is a polynucleotide. Generally oligonucleotides will have fewer than 250 nucleotides, in some embodiments, between 4-200, e.g., 10-150 nucleotides.

“Clonal” copies of a polynucleotide means the copies are identical in sequence. In some embodiments, there are at least 100, 1000, 10⁴ or more clonal copies of oligonucleotides in linked to a bead.

A “3′ capture sequence” on an oligonucleotide refers to the 3′ most portion of an oligonucleotide. The capture sequence can be as few as 1-2 nucleotides in length but is more commonly 6-12 nucleotides in length and in some embodiments is 4-20 or more nucleotides in length. The capture sequence can be completely complementary to a target nucleic acid (e.g., the 3′ end of the target nucleic acid), though as will be appreciated in some embodiments and certain conditions, 1, 2, 3, 4, or more nucleotides may be mismatched while still allowing the 3′ capture sequence of an oligonucleotide anneal to the target nucleic acid. In other embodiments, conditions can be selected such that only completely complementary sequences will anneal. The 3′ capture sequence can be a random sequence, a poly T or poly A sequence, a target-specific sequence, or a universal sequence. For example, in embodiments in which a transposon (e.g., a modified Tn5 such as tagmentase) inserts an end sequence to sample nucleic acids, the capture 3′ end sequence can be complementary or identical to the added end.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., Monoclonal Antibodies and Cancer Therapy, pp. 77-96. Alan R. Liss, Inc. 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, (1992)).

The antibody binds to an “epitope” on the antigen. The epitope is the specific antibody binding interaction site on the antigen, and can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope). The same is true for other types of target molecules that form three-dimensional structures.

The term “specifically bind” refers to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds α ταργεσ πργειν will typically bind to σαργεσ πρoσειν with at least a 2-fold greater affinity than a non-target (e.g., other cellular proteins).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

### BRIEF DESCRIPTION OF THE DRAWINGS

FIGS. 1A-1E depict barcoding with barcode bead plugs: 1A) Core empty gel beads, or nanovials, are allowed to settle onto a 2D surface. 1B) Cells are added to the solution and enter the nanovials through settling and/or centrifugation. They attach to the basement of the nanovials 1C) Barcode gel beads are added to the solution and enter the nanovials. They also bind to the interior of the nanovial. 1D) Cell lysis and reverse transcription reagents are added to the plug-cell-nanovial complex. The reverse transcriptase has sufficiently low molecular weight to support diffusion throughout the hydrogel complex comprising the nanovial and the barcode gel bead. RNA/DNA that is released from lysing cells have sufficiently high molecular weight to be trapped within the complex. 1E) The barcode oligo on the barcode bead is used to prime RNA for reverse transcription and barcoded cDNA generation. The sample is prepped for NGS and sequenced.

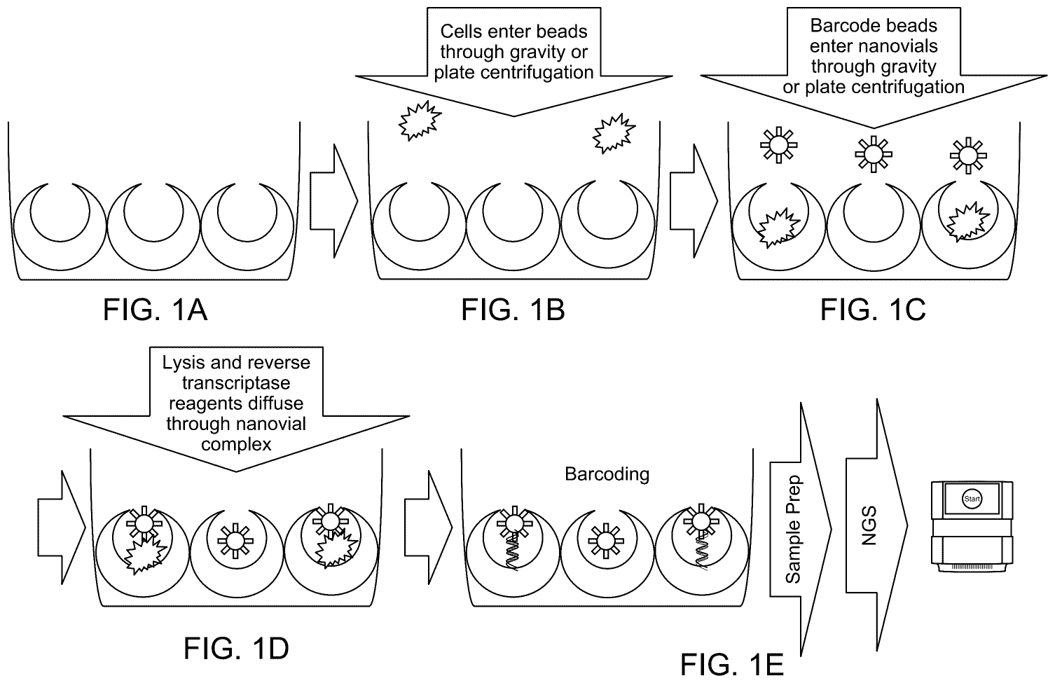

FIGS. 2A-2E depict barcoding with swollen barcode bead plugs: 2A) Core empty gel beads, or nanovials, are allowed to settle onto a 2D surface. 2B) Cells are added to the solution and enter the nanovials through settling and/or centrifugation. They attach to the basement of the nanovials. 2C) Barcode gel beads are added to the solution and enter the nanovials. They also bind to the interior of the nanovial. 2D) Gel beads swelling solution is added. The gel beads swell to create a plug at the orifice of the nanovial. Cell lysis and reverse transcription reagents are applied. The reverse transcriptase has sufficiently low molecular weight to support diffusion throughout the complex. RNA/DNA that is released from lysing cells have sufficiently high molecular weight to be trapped within the complex. 2E) The barcode oligo on the barcode bead is used to prime RNA for reverse transcription and barcoded cDNA generation. The sample is prepped for NGS and sequenced.

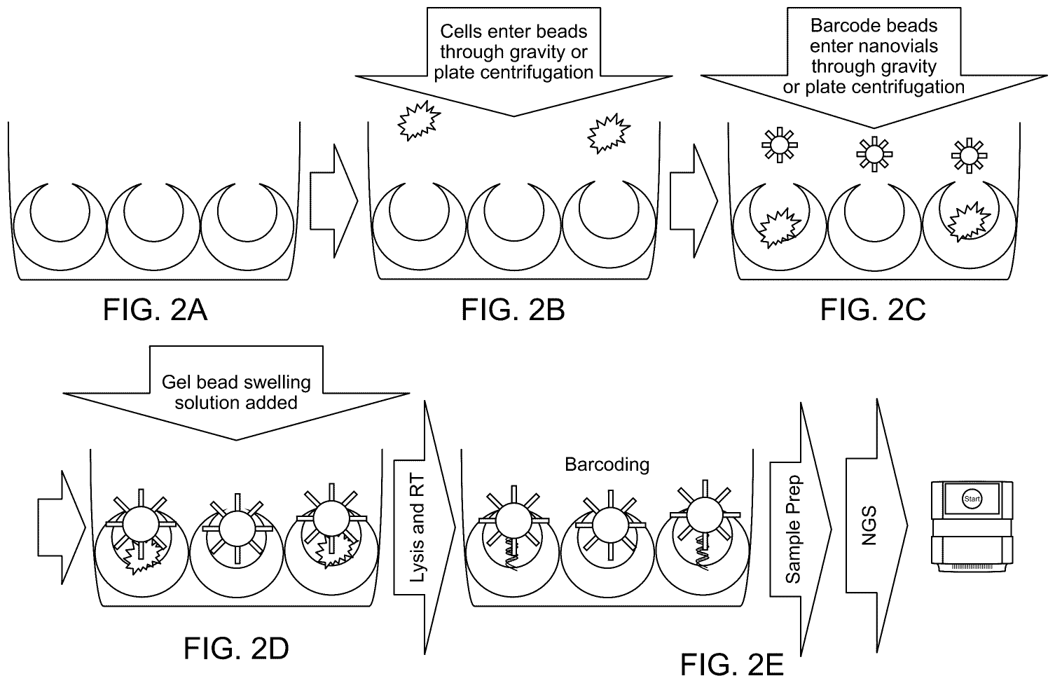

FIGS. 3A-3E depict barcoding with multiple barcode bead plugs: 3A) Core empty gel beads, or nanovials, are allowed to settle onto a 2D surface. Cells are added to the solution and enter the nanovials through settling and/or centrifugation. They attach to the basement of the nanovials. 3B) Cells enter beads through gravity or plate centrifugation. 3C) A high concentration of barcode gel beads are added to the solution and enter the nanovials. They also bind to the interior of the nanovial. 3D) Cell lysis and reverse transcription reagents are added to the plug-cell-nanovial complex. The reverse transcriptase has sufficiently low molecular weight to support diffusion throughout the complex. RNA/DNA that is released from lysing cells have sufficiently high molecular weight to be trapped within the complex. 3E) The barcode oligo on the barcode bead is used to prime RNA for reverse transcription and barcoded cDNA generation. The sample is prepped for NGS and sequenced.

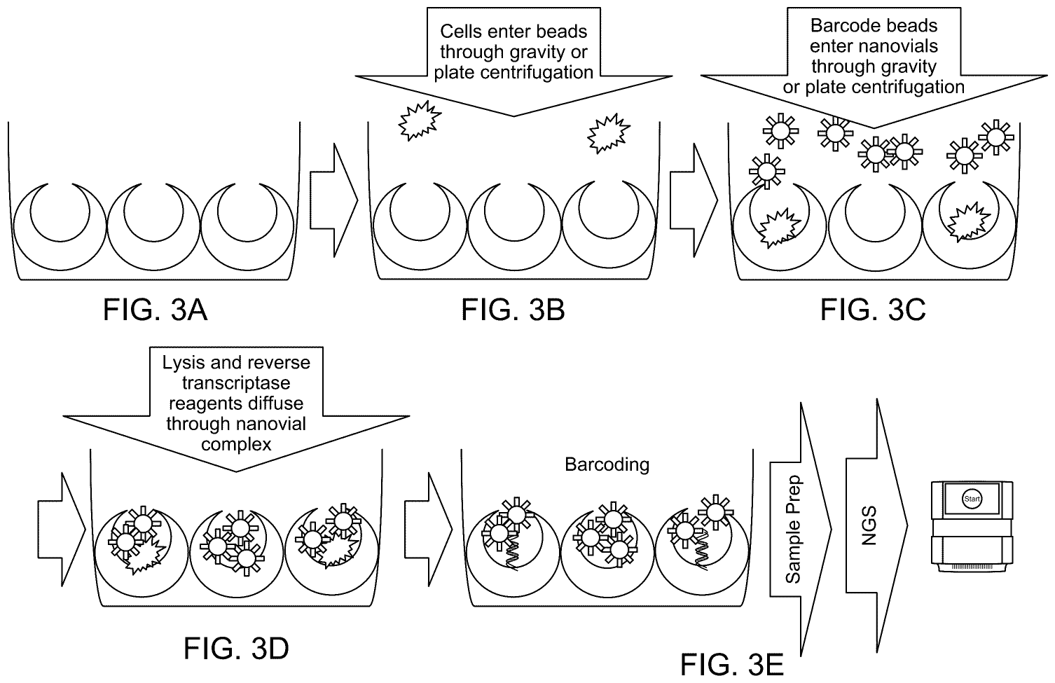

FIGS. 4A-4D depict barcoding with barcode beads and oil. 4A) Cells and beads enter arrayed nanovials through gravity settling or centrifugation. Although more than one gel bead per nanovial is depicted, the geometry of the nanovial and gel bead can be such that only a single gel bead enters the nanovial. 4B) Oil is added to the mix and the nanovials containing cells and beads solution are agitated to form droplets around the nanovials as shown in (4C). 4D) The mixture is placed into a thermocyler for barcoding, the barcoded nucleic acid sample is prepped and NGS ensues.

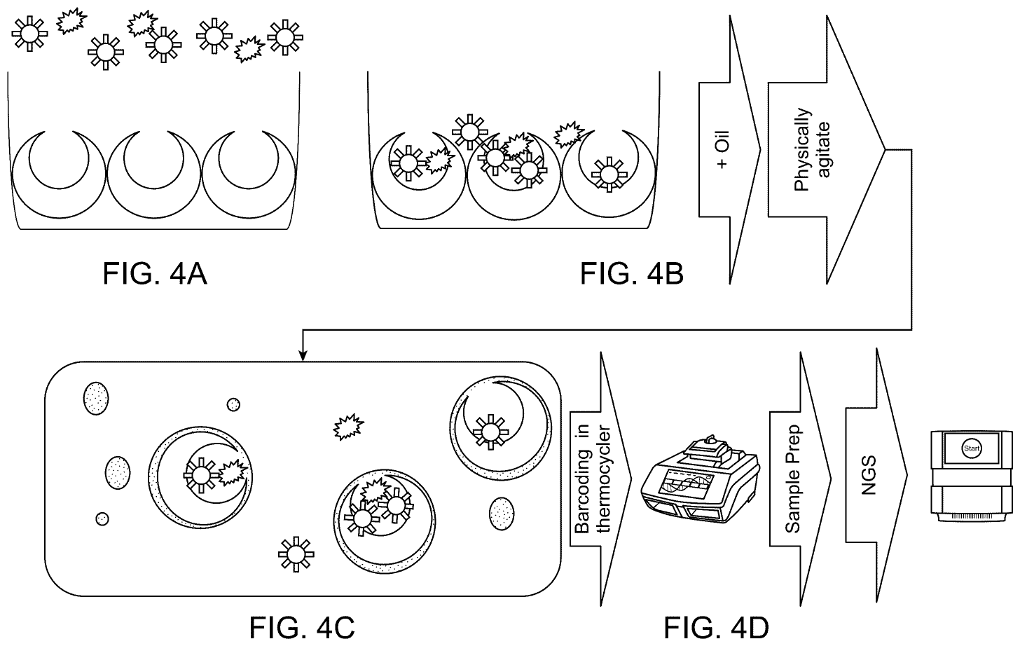

FIGS. 5A-5F depict a multistep process with swollen barcode bead plugs: 5A) Core empty gel beads, or nanovials, are allowed to settle onto a 2D surface. 5B) Cells are added to the solution and enter the nanovials through settling and/or centrifugation. They attach to the basement of the nanovials. 5C) Barcode gel beads are added to the solution and enter the nanovials. They also bind to the interior of the nanovial. 5D) Gel beads swelling solution is added. The gel beads swell to create a plug at the orifice of the nanovial. 5E) Protein degradation reagents are added to denature and destroy all proteins including histones bound to DNA. During this step, high molecular weight nucleic acids cannot diffuse out of the core where the cell used to be. Upon completion of protein destruction, protein degradation solution is deactivated and/or washed out. 5F) Barcoding enzymes are added followed by barcoding, sample prep and NGS.

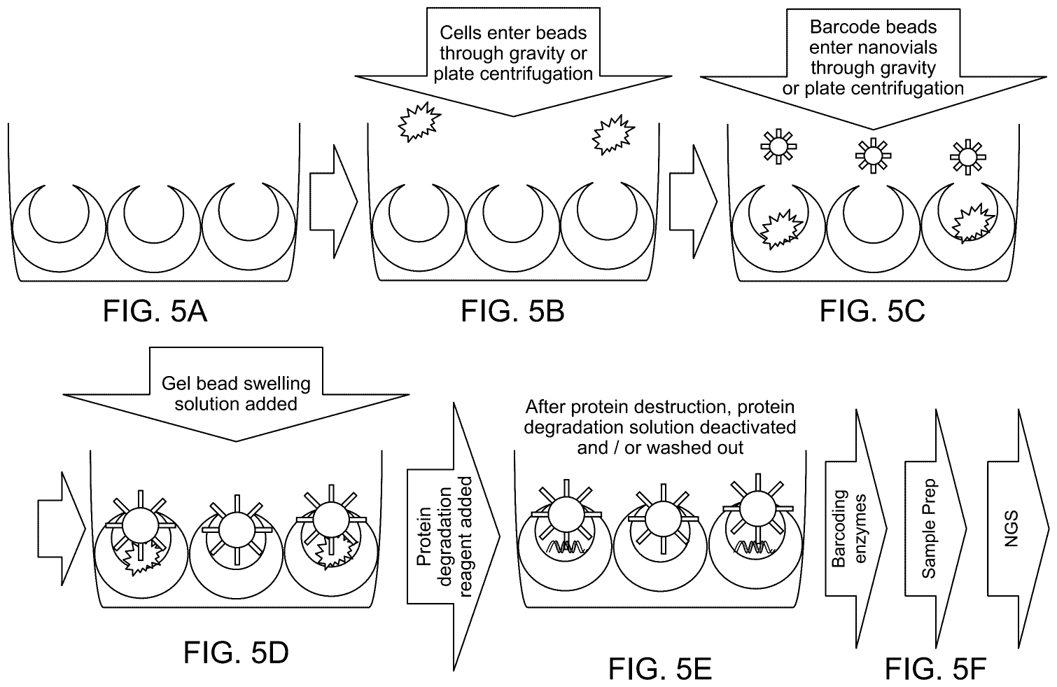

FIG. 6 depicts layers of a possible nanovial having from bottom to top as depicted, a PEG phase layer, a gelatin layer and oligonucleotides linked to the gelatin layer. The right side of the figure depicts an embodiment in which a three-arm PEG spacer is linked to gelatin.

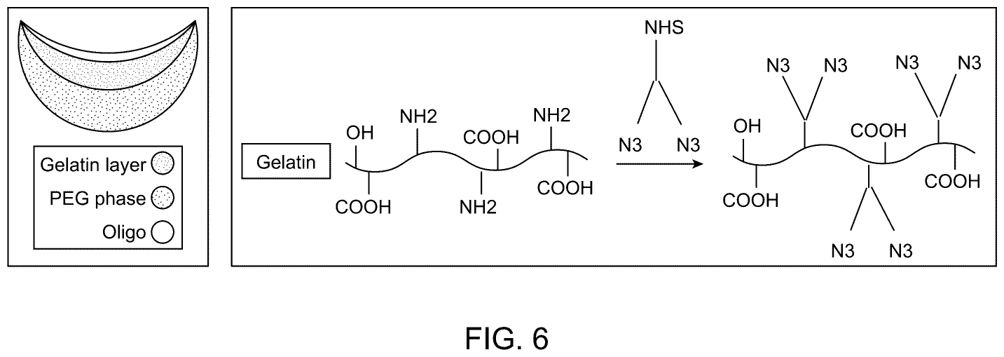

FIG. 7 depicts use of click chemistry to attach the cell-barcoding oligonucleotides to the nanovial. As shown, linkage of an NHS-PEG-azide three-arm linker (linked to gelatin as shown in FIG. 6) to a DBCO-modified oligonucleotide can occur via a click chemistry reaction.

### DETAILED DESCRIPTION OF THE INVENTION

### Introduction

It has been determined that openings in hollow nanovials, which can contain introduced cells, can be subsequently occluded by introduction of hydrogel beads into the openings, functionally rendering the nanovials into partitions that retain large biological molecules such as RNA and DNA but into which reagents can be diffused to perform a number of molecular biology processes. For example, the cells in the occluded nanovials can be lysed and nucleic acids from lysed cells can be linked to barcoded oligonucleotides, which can be present in the nanovials or that can be introduced with (e.g., linked to) the hydrogel beads. Thus, for example, single cell nucleotide sequencing methods that have in the past been performed in droplets can instead be performed in occluded nanovials as described herein. Examples of techniques that can be performed in the occluded nanovials instead of other types of partitions include ATAC-seq and RNA-seq, among others. A similar method can be applied to microwells, for example occluding microwell openings with hydrogel beads as explained herein with regard to nanovial openings. Similarly, the methods using occluded nanovials can be equally applied to occluded microwells.

Cells can be introduced into hollow nanovials or microwells as desired. For example, in some embodiments, the cells are mixed with the hollow nanovials. In some embodiments, the hollow nanovials are a crescent (e.g., “C”) shape and are allowed to settle onto a planar surface. Because of the shape and off-center weight of the hollow nanovials their openings will face up and thus cells can be allowed to settle into the openings of the hollow nanovials. In some embodiments, the size of the openings and cavity of the nanovials is such that on average the cavity can only accommodate one cell. In some embodiments, a majority (e.g., at least 50, 60, 70, 80, or 90% of the beads) contain only one cell, or optionally one or zero cells. This is achieved by loading on average 0.05-0.1 cells per nanovial. If binding moieties (e.g., biotin or streptavidin) are present on the nanovials, the cells can bind to the nanovial provided the respective binding partner is located on the outer membrane of the cell. Coating the basement of the nanovial with one or more integrins or another cell-binding agent is another way to provide a capture patch to bind cells that enter the nanovial cavity. In some embodiments, cell capture is irreversible. In some embodiments the binding moieties, and thus cell capture, are reversible.

Nanovials are generally hollow hydrophilic beads with an opening for entry and exit of samples and reagents. Exemplary hollow nanovials are described in, for example, de Rutte, et al., bioRxiv Sep. 18, 2020 (https://doi.org/10.1101/2020.03.09.984245), de Rutte, ACS Nano (2022) https://doi.org/10.1021/acsnano.1c11420 and U.S. Patent Publication No. 2021/0268465 (describing the hollow beads as “dropicles” or “drop-carrier particles”). The hollow beads can have a diameter, for example, of between about 10 μm and about 500 μm, e.g., between 20 μm and 200 μm, e.g., between 40 and 20 μm. In some embodiments, the hollow beads are crescent shape such that they have a tendency to settle under gravity with their openings up. This occurs because the center of gravity is located off center in the opposite direction to the nanovial opening. The beads include a void volume or cavity therein. The void or cavity delimits the three-dimensional volume that holds at least a portion of a dispersed phase solution or fluid (e.g., aqueous phase). Exemplary fluid volumes held within the void or cavity can be for example about 100 fL-10 nL. A length dimension (e.g., diameter for spherical void) of the void or cavity within a bead can be several microns, e.g., more than about 5 μm and less than about 250 μm.

In some embodiments, hollow nanovials are generated by flowing two phases to form an emulsion resulting in the hollow nanovials. Microgel beads containing cavities can be fabricated, for example, using aqueous two-phase systems (ATPS) combined with droplet microfluidics. See, e.g., U.S. Patent Publication No. US 2021/0268465; S. Ma, et al. Small. 8, 2356-2360 (2012); B. D. Fairbanks, et al., A versatile synthetic extracellular matrix mimic via thiol-norbornene photopolymerization. Adv. Mater. (2009). In some embodiments one phase of an aqueous two-phase systems comprises a crosslinkable component, while the other phase does not contain the crosslinkable component. In some embodiments a crosslinkable PEG phase and dextran phase are co-flowed in a microfluidic droplet generator device along with a third oil phase containing surfactant to generate mixed aqueous emulsions in which a uniform fraction of PEG phase and dextran phase is present in each droplet suspended in an oil phase. In other embodiments, instead of dextran, gelatin is used. For example, in some embodiments a crosslinkable PEG phase and gelatin phase are co-flowed in a microfluidic droplet generator device along with a third oil phase containing surfactant to generate mixed aqueous emulsions in which a uniform fraction of PEG phase and gelatin phase is present in each droplet suspended in an oil phase. See, e.g., Lee, ACS Nano 2022, 16, 38-49. In some embodiments, separate prepolymer solutions of poly(ethylene glycol) diacrylate (PEGDA) and poly(propylene glycol) diacrylate (PPGDA) are co-flowed, shaped in the streams to the desired cross-sectional morphology in a microchannel flow, and then photocrosslinked. See, e.g., Wu et al., Sci. Adv. 2020. The same reference shows that other continuous phases that are immiscible with water, including poly(dimethylsiloxaneco-diphenylsiloxane) (PSDS), PPG, decanol, and toluene can also used.

The nanovials (or microwells), the hydrogel beads, or both, can be linked to oligonucleotides comprising barcodes identifying the nanovial and/or hydrogel, respectively, which act as partition-specific barcodes. In some embodiments, the hollow nanovials are linked or otherwise associated with a plurality of clonal cell-barcoding oligonucleotides having a 3′ capture sequence. In some embodiments, the cell-barcoding oligonucleotides can be linked as desired to the hydrogel beads (see below). Methods of linking the cell-barcoding oligonucleotides to the hydrogel beads or nanovials will depend on the composition of the beads. In some embodiments, the 5′ end of the cell-barcoding oligonucleotides is linked to the beads or the cell-barcoding oligonucleotides are otherwise linked such that the 3′ capture sequence is available for hybridization to complementary sequences.

In some embodiments, the cell-barcoding oligonucleotides is linked to the nanovials via a spacer, allowing the barcoding oligonucleotides to more available to anneal to complementary sequences. In some embodiments, the spacer allows for more than one barcoding oligonucleotide to be linked to the spacer, allowing for a greater density of barcoding oligonucleotides on the nanovial. For example, in some embodiments, a 3-arm polyethylene glycol (PEG) with a terminal NHS group to the gelatin cavity of a nanovial can be used, and will double the functional groups on the cavity surface for oligonucleotide attachment. An exemplary three-arm PEG spacer is shown in formula I

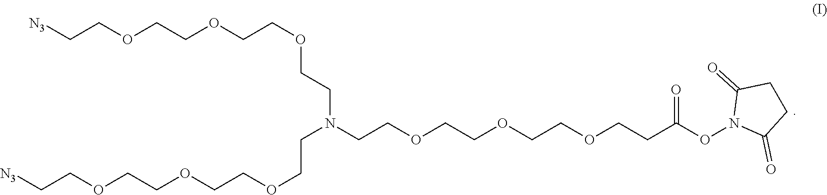 [SMILES: [N-]=[N+]=NCCOCCOCCOCCN(CCOCCOCCOCCN=[N+]=[N-])CCOCCOCCOCCC(=O)ON1C(=O)CCC1=O]

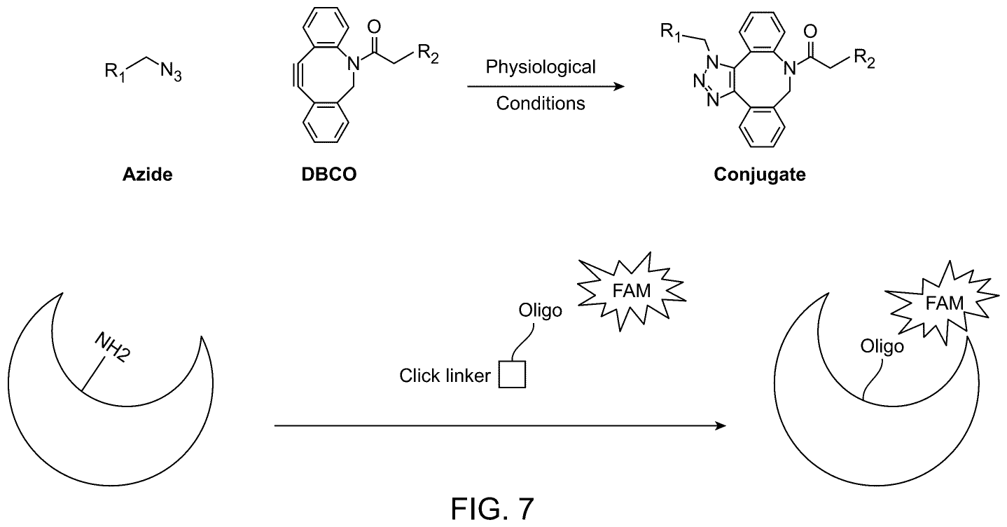

In some embodiments, click chemistry can be used to attach the cell-barcoding oligonucleotides to the nanovial or beads. For example, NHS-PEG-azide can be attached to amine (NH₂) moieties on the cavity or elsewhere on a nanovial, followed by attachment of DBCO modified oligonucleotides to the azide groups via click chemistry. See, FIGS. 7-8. Click chemistry involves an azide alkyne Huisgen cycloaddition reaction, wherein in some embodiments described herein, a first oligonucleotide having an end alkyne moiety (for example but not limited to, DBCO-modified) is reacted with a spacer having an end azide moiety and promoting or catalyzing a reaction between the moieties to covalently-link the two oligonucleotides. In some embodiments, the azide-alkyne Huisgen cycloaddition is a 1,3-dipolar cycloaddition between an azide and a terminal or internal alkyne to give a 1,2,3-triazole. The reaction can in some embodiments be catalyzed by copper (Copper(I)-catalyzed azide-alkyne cycloaddition (CuAAC)) or promoted by a strained difluorooctyne (DIFO) (Strain-promoted azide-alkyne cycloaddition (SPAAC)) for example. See, e.g., Chemical Reviews: Click Chemistry, Jun. 23, 2021, Volume 121, Issue 12, Pages 6697-7248, including for example, Fantoni et al., “A Hitchhiker's Guide to Click-Chemistry with Nucleic Acids” Chem. Rev. 2021, 121, 7122-7154. The click ligation can be proceeded in some embodiments, by an incubation at degrees C., for example, for 2 hours in the presence of Vitamin C, Cu(II)-TBTA, MgSO₄, THPTA, and DMSO.

In other embodiments, partition-specific barcoding oligonucleotides are not linked or associated with the hollow nanovials, and in some of these embodiments, the oligonucleotides can be delivered to the cavity within the hollow nanovials via hydrogel beads as described more below.

In some embodiments, sets of the hollow nanovials can be linked to or otherwise associated with identical hashing oligonucleotides, wherein different sets have different hashing oligonucleotides, allowing for different sets of nanovials to be tracked via the hashing barcodes in the hashing oligonucleotides. These would be used, for example, for cell hashing, e.g., as described in, e.g., Stoeckius et al., Genome Biology (2018) 19:224. The hashing oligonucleotides can comprise, for example, a 5′ PCR handle sequence, allowing for a universal primer binding sequence, for example, a hashing barcode sequence that identified a set of nanovials from other nanovials, and (i) a 3′ sequence that is complementary to a 3′ sequence downstream of the barcode segment in the partition-specific barcoding oligonucleotide and/or (ii) a splint sequence, allowing for later attachment of the hashing oligonucleotides to partition-specific barcoding oligonucleotides. In some embodiments, the attachment can comprise hybridization of the hashing oligonucleotide directly to the partition-specific barcoding oligonucleotides with or without primer templated DNA synthesis. In some embodiments, the attachment can comprise hybridization of the hashing oligo indirectly to the partition-specific barcoding oligonucleotide through hybridization to a splint oligonucleotide with or without ligation. In some embodiments, the attachment can comprise linking the hashing oligonucleotide to amplicons formed from the partition-specific barcoding oligonucleotides and target nucleic acids from lysed cells through primer templated hybridization extension and/or ligation. In some embodiments, the nanovials can be linked to hashing oligonucleotides and partition-specific barcoding oligonucleotides.

Hashing oligonucleotides can be used to provide a label for sets of nanovials. Sets of nanovials can receive different samples for example, allowing the hashing oligonucleotides to label different samples, which can later be mixed and tracked via barcoding. In some embodiments, sets of nanovials can receive different aliquots of cells from the same sample, for example, allowing the hashing oligonucleotides to label different aliquots from the same sample, which can later be mixed and tracked via barcoding. In some embodiments, sets of nanovials can receive different samples and different aliquots from the same sample, which can later be mixed and tracked via barcoding. In some embodiments, cells can be introduced into nanovials comprising hashing oligonucleotides, different sets with different hashing oligonucleotides can be combined and occluded as described herein (the order can be occlusion and combination or combination and occlusion). Partition-specific barcoding of cell nucleic acids can then be performed and hashing oligonucleotides can be linked to the resulting partition-specific barcoded sample nucleic acids. Nucleotide sequences can later be used to track sequences from different sets of nanovials via the hashing oligonucleotide and specific partitions can be tracked via the partition-specific barcodes.

Any type of cells can be introduced into the hollow nanovials (or microwells). In some embodiments, the cells are eukaryotic cells, e.g., human, mouse, rat or other mammalian cells. In some embodiments, the cells are immune cells, e.g., including but not limit to hybridomas, T-cells or B-cells. In some embodiments the cells are bacteria or fungi or plant cells.

Following introduction of cells into the hollow nanovials (or microwells), one can form an occlusion in the opening in the beds, thereby preventing diffusion of the cells from the nanovials (or microwells). For example, whereas cells and cell products from lysed cells can readily diffuse from nanovial openings, once the openings are occluded the cells and cell products will not substantially escape from the nanovials, allowing for use of the occluded nanovials as partitions, without the need for droplet, microwell or other partitoning technology in some embodiments. This can be achieved, for example, by introduction of one or more hydrogel beads into the openings in the hollow nanovials thereby preventing exit of the cells from the cavity in the hollow nanovials. In some embodiments, introduction of the hydrogel beads into the openings is sufficient to occlude the opening, not releasing for example, intact cells in the nanovial. The hydrogel beads can be introduced into the nanovials as desired, for example, beads can be allowed to settle into the nanovials by gravity or through centrifugation. In other embodiments, following introduction of the hydrogel beads into the opening in the nanovials, one or more agent or change of conditions (e.g., change of temperature) is introduced to cause the hydrogel beads to swell, thereby reducing their ability of escaping from the openings and filling the gaps between the interior of the nanovial and the gel bead surface. In some embodiments, the hydrogel beads comprise polyacrylamide and swelling of the beads can be induced by contacting the beads with ethanol.

In some embodiments, the hydrogel bead can comprise a component, which when cleaved, results in swelling, thereby occluding the opening of the nanovial. For example, in some embodiments, the bead comprises a component that is lysed upon contact with a reducing agent. An example of such a component is for example polyacrylamide that is cross-linked with Bis-acrylylcystamine. Exemplary reducing agents can be applied to cleave disulfide bonds in the linker. Exemplary reducing agents can include, but are not limited to dithiothreitol (DTT), or tris(2-carboxyethyl)phosphine (TCEP) or other reducing agents.

In other embodiments, the hydrogel comprises a component that is photocleavable. Upon exposure to light, or of light of a wavelength that photocleaves the component, the bead will swell such that when present in an opening of a nanovial will occlude the nanovial opening. In some embodiments, a photocleavable element can be incorporated into the hydrogel bead matrix, for example, through one or more acrylate groups. In some embodiments, the photocleavable component is acrylate-functionalized dextran. In some embodiments, the bead comprises acrylaminde or other hydrogel component comprising a photocleavable cross-linker such as, for example, ortho-nitrobenzyl (o-NB) bis-acrylate.

In addition, or alternatively, low salt buffers or pure water can be used to swell the beads whereas high salt buffers can be used to shrink the beads, which for example, may be induced to allow the beads to enter the nanovials. An exemplary high salt buffer that can shrink the hydrogel gel bead is for example, 100 mM MgCl₂. An exemplary low salt buffer that can swell a bead is for example 10 mM Tris 1 mM EDTA. In another embodiment, a polyethylene glycol (PEG) solution (e.g., 15% w/v PEG 6000) will also shrink the beads. In this case, the beads will subsequently swell if the PEG solution is replaced with one that does not contain PEG or contains a lower concentration of PEG.

In some embodiments, the occlusion prevents escape of cellular products as well as the cells themselves. For example, large molecules such a polynucleotides and proteins will in general be unable to significantly diffuse from the cavity within the occluded nanovial. Optionally, if the escape of particular cellular products and/or nucleic acid species are desired, those substrates can initially be cleaved to generate lower molecular weight products that diffuse through the nanovial—gel bead complex. One can measure for successful occlusion of the nanovial in any way that detects the ability of the nanovial to retain molecules compared to open nanovials (e.g., not receiving a hydrogel bead). In some embodiments, a species mixing experiment can be performed to measure occlusion. For example, mouse and human cells can be loaded in the nanovials. Upon barcoding, sample preparation and next generation sequencing, the amount of nucleic acid from the species not contained within the nanovial can be a measure of how well the plug—nanovial complex prevents movement of the nucleic acids from one nanovial to another. This is referred to as a “cell purity” metric.

Hydrogel beads of various composition have been described previously. It is recognized that hydrogels are hydrophilic and as such in some embodiments, the “nanovial” and the “hydrogel bead” may be composed of the same materials, whereas in other embodiments, the two are composed of different materials. In any case, to maintain clarity between hollow beads containing the cells and the beads that occlude the openings, the terms “hollow nanovial” or “nanovial” is used to describe the former whereas “hydrogel bead” is used to describe the latter.

Hydrogel beads can be composed of various materials. Exemplary hydrogel beads can be, for example, agarose hydrogel beads or polyacrylamide hydrogel beads. In other embodiments, the hydrogels comprise, for example, polystyrene, silica, polymethylmethacrylate, alginate, poly ethylene glycol (PEG), nylon, and/or other polymers, with or without crosslinking. Other hydrogels include, but are not limited to, those described in, e.g., U.S. Pat. Nos. 4,438,258; 6,534,083; 8,008,476; 8,329,763; U.S. Patent Appl. Nos. 2002/0,009,591; 2013/0,022,569; 2013/0,034,592; and International Patent Publication Nos. WO/1997/030092; and WO/2001/049240. In general, the size of the hydrogel beads can be selected to allow for the hydrogel bead to enter openings in the hollow nanovials but be large enough to occlude the openings as descried herein. The hydrogel beads can also be sensitive to shrinking and swelling agents as described herein. The percent matrix of the hydrogel determines the porosity of the hydrogel and can be chosen to prevent the movement of target molecules outside the nanovial gel bead complex, while allowing free diffusion of buffers and low molecular weight proteins. Hydrogels that are greater than 3% hydrogel material generally trap high molecular weight nucleic acids but allow for the diffusion of low molecular weight enzymes, ions, and detergents.

In some embodiments, a population of hydrogel (e.g., acrylamide crosslinked with bis-acrylamide) functionalized beads (e.g., containing functionalized oligonucleotides linked to the hydrogel beads) that are sized slightly (e.g., 0.5%-25%) larger than an opening in a hollow nanovial, can be exposed to buffers (e.g., 10 mM MgCl₂) containing materials that produce smaller equilibrium sizes of functionalized beads, thereby shrinking the majority of to a size smaller than the hollow nanovial opening, loading the functionalized beads into the hollow nanovial openings, and then changing the surrounding buffer (e.g., to a lower salt buffer, such as tris EDTA buffer, etc.) to produce swelling but with retention of the functionalized beads in the hollow nanovial openings. Such a process effectively closes off the hollow nanovial opening with one or more semipermeable functionalized beads.

In some embodiments, in addition to occluding openings in the nanovials (or microwells), the hydrogel beads can also deliver oligonucleotides to the cavity of the nanovials (or microwells), e.g., to be attached to target cellar nucleic acids in the cavity. Oligonucleotides can be linked to beads as desired. The solid support surface of the bead can be modified to include a linker for attaching barcode oligonucleotides. The linkers may comprise a cleavable moiety. Non-limiting examples of cleavable moieties include a disulfide bond, a dioxyuridine moiety, and a restriction enzyme recognition site. Methods of linking oligonucleotides to beads are described in, e.g., WO 2015/200541. In some embodiments, the oligonucleotide configured to link a hydrogel bead to the barcode is covalently linked to the hydrogel. Numerous methods for covalently linking an oligonucleotide to one or more hydrogel matrices are known in the art. As but one example, aldehyde derivatized agarose can be covalently linked to a 5′-amine group of a synthetic oligonucleotide.

Optionally, the plurality of nanovials, many but not all of which contain cells, can be enriched for beads that contain cells. This can be achieved, for example where the cells emit a fluorescent signal, allowing one to sort the beads via FACS. Alternatively, methods such as differential sedimentation or centrifugation can be used to enrich for higher density cell-containing beads. The fluorescent signal may accumulate throughout the nanovial, throughout the gel bead and/or in some particular location through the nanovial gel bead complex such as at basement of the nanovial opening.

Following occlusion of the openings in the hollow nanovials (or microwells), the cells within the nanovials (or microwells) can be lysed. Lysis can be induced by temperature or introduction of one or more reagents that result in lysis of the cells. Exemplary lysis agents can comprise, for example, detergents (e.g., SDS), protein denaturants and/or proteases. Because of the nature of the nanovial and the occluding hydrogel bead, small molecules can diffuse into the cavity of the nanovial. Thus, lysis reagents can be incubated with the nanovials, allowing for the lysis reagents to diffuse into the cavity of the beds where the cells reside. In some embodiments, the protein denaturant comprises guanidine. Protein denaturants and/or proteases are useful, for example, to remove histones from chromosomal DNA when cellular DNA is the target cellular nucleic acid. Optionally following lysis, the nanovials can be submitted to one or more wash/rinse steps to diffuse away the lysis reagents after lysis has occurred, leaving larger molecules from the lysed cells in the cavity of the nanovials.

Large molecules, such as DNA and RNA (e.g., nucleic acid species that are greater than 1 kbp (650 KDa)), from the lysed cells will substantially remain in the cavity of the nanovials, allowing for molecular methods to be applied to the nucleic acids, substantially isolated from nucleic acids within separate nanovials. On the other hand, smaller molecules, including for example polymerases, can be diffused into the cavity of the nanovial allowing for a variety of molecular reactions to be initiated. Thus, for example, oligonucleotides present in the cavity of the nanovial (either for example introduced linked to the nanovial or delivered by the hydrogel bead) can be linked to one or more nucleic acid from the cells. The nucleic acids can be for example, RNA or DNA.

The oligonucleotides can be used to perform primer extension or other hybridization-based reactions. In some embodiments, the 3′ end sequences of the oligonucleotides anneal directly to the target nucleic acids. For example, if the target nucleic acids are mRNA, the 3′ end sequence can be a poly dT sequence (e.g., 6-20 contiguous dT nucleotides), or the 3′ end can include randomer (e.g., random sequences of 6-10 or more nucleotides) to randomly prime targets, or the 3′ end sequences can be gene-specific to specifically amplify one or more target nucleic acids. In some embodiments, the 3′ sequence of the oligonucleotides anneals to a universal sequence on the target nucleic acids. For example, tagmentation can result in insertion of an adaptor sequence to the end of fragmented nucleic acids and the 3′ end can be reverse complementary to the adaptor sequence. In some embodiments, the oligonucleotides can be ligated to cellular DNA or used as primers in a primer extension reaction using a DNA polymerase and the cellular DNA as a template to form a DNA molecule comprising the oligonucleotide sequence as well as at least a part of the cellular DNA. In any of these embodiments, the oligonucleotides can be linked at their 5′ end to a solid support (e.g., either bead) or can have been released from the support into solution within the cavity prior to linking to a cellular nucleic acid.

In any of the embodiments described herein, the oligonucleotides linked to the beads can comprise a plurality of clonal copies of one or more barcode nucleotide sequences. In some embodiments, the oligonucleotides include a barcode sequence that is unique to the bead to which it is attached and thus can be used to distinguish oligonucleotides from different beads, e.g., after the oligonucleotides are released and used to generate sequencing reads. For example, most or substantially all of the cellular nucleic acids linked to the oligonucleotides will include the same barcode sequence, or where more than one hydrogel bead has introduced more than one oligonucleotide barcode (one clonal set of barcodes per bead in the cavity) there can be two or several different barcode sequences linked to different nucleic acids from the same cells, requiring deconvolution downstream to either remove or decipher that differently barcoded nucleic acids originated from the same cell. Additional barcodes, such as but not limited to, unique molecule identifiers (UMIs) or sample-specific barcodes can also be included in the oligonucleotide sequence and thus be linked the cellular nucleic acids.

Optionally, the occluded nanovials can be introduced into a droplet, or other partition, for further manipulations. In other embodiments, the occluded nanovials are not introduced into droplets or other partitions and the occluded nanovials themselves are employed as partitions.

Once molecular reactions as described above have been completed (for example barcodes have been attached to the cellular nucleic acids) in the hollow nanovial (or microwells), the contents of the nanovials (or microwells) can be combined into a bulk solution. In some embodiments, the hydrogel bead(s) that occlude the opening in the nanovial (or microwell), or the nanovial itself, or both, are digested or otherwise degraded to allow for release of the contents of the nanovials (or microwells). This can be achieved for example, by applying heat or one or more reagent that degrades or dissolves the hydrogel bead or nanovial or both, releasing the contents. In some embodiments, the hydrogel bead comprises polyacrylamide and N,N′-Bis(acryloyl)cystamine, which is a reversible cross-linker that dissolves when reacted with reducing agents such as dithiothreitol (DTT) or tris(2-carboxyethyl)phosphine (TCEP) or other reducing agents. In some embodiments, the solvent around the nanovials is changed to cause the hydrogel beads to shrink and thereby release the nanovial contents. As noted above, hydrogel beads can be induced to shrink in the presence of, for example, ethanol, PEG (e.g., 15%) and/or 100 mM MgCl₂.

The nucleic acids can then be nucleotide sequenced. Once the nucleic acids have been tagged with the oligonucleotides, the tagged nucleic acids can be prepared for nucleotide sequencing as desired. For example, universal priming sequences can be added on both ends of the tagged sequences (one universal priming sequence each end). Any method of nucleotide sequencing can be used as desired so long as at least some of the DNA segments sequence and the barcode sequence is determined. Methods for high throughput sequencing and genotyping are known in the art. For example, such sequencing technologies include, but are not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety.

Exemplary DNA sequencing techniques include fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the present technology provides parallel sequencing of partitioned amplicons (PCT Publication No. WO 2006/084132, herein incorporated by reference in its entirety). In some embodiments, DNA sequencing is achieved by parallel oligonucleotide extension (See, e.g., U.S. Pat. Nos. 5,750,341; and 6,306,597, both of which are herein incorporated by reference in their entireties). Additional examples of sequencing techniques include the Church polony technology (Mitra et al., 2003, Analytical Biochemistry 320, 55-65; Shendure et al., 2005 Science 309, 1728-1732; and U.S. Pat. Nos. 6,432,360; 6,485,944; 6,511,803; herein incorporated by reference in their entireties), the 454 picotiter pyrosequencing technology (Margulies et al., 2005 Nature 437, 376-380; U.S. Publication No. 2005/0130173; herein incorporated by reference in their entireties), the Solexa single base addition technology (Bennett et al., 2005, Pharmacogenomics, 6, 373-382; U.S. Pat. Nos. 6,787,308; and 6,833,246; herein incorporated by reference in their entireties), the Lynx massively parallel signature sequencing technology (Brenner et al. (2000). Nat. Biotechnol. 18:630-634; U.S. Pat. Nos. 5,695,934; 5,714,330; herein incorporated by reference in their entireties), and the Adessi PCR colony technology (Adessi et al. (2000). Nucleic Acid Res. 28, E87; WO 2000/018957; herein incorporated by reference in its entirety).

Typically, high throughput sequencing methods share the common feature of massively parallel, high-throughput strategies, with the goal of lower costs in comparison to older sequencing methods (See, e.g., Voelkerding et al., Clinical Chem., 55: 641-658, 2009; MacLean et al., Nature Rev. Microbiol., 7:287-296; each herein incorporated by reference in their entirety). Such methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and platforms commercialized by VisiGen, Oxford Nanopore Technologies Ltd., Life Technologies/Ion Torrent, and Pacific Biosciences, respectively.

The practice of the present invention can employ conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999-2010) Current Protocols in Molecular Biology, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag; Sambrook et al., Molecular Cloning: A Laboratory Manual (2nd ed.) (1989).

In some embodiments, once the sequencing reads are generated, the sequencing reads can be deconvoluted to pool sequencing reads having different barcodes that nevertheless originated from the same partition. Thus, in some embodiments, one can use the methods described above involving determining the percent of identical fragments in common and combining sample reads to assume all such reads are from the same partition when the percent of identical fragments exceeds a threshold, for example as described in U.S. Patent Publication 2020/0056231. Other methods for deconvoluting the sequencing reads (determining whether reads come from the same or different partitions) can include, for example, those described in PCT WO2017/120531. For example, in some embodiments the method can involve providing in a partition a substrate comprising a barcode sequence or repeating clonal barcode sequences; and in the partition, associating a first particle conjugated to oligonucleotide primers comprising a first barcode sequence and a second particle conjugated to oligonucleotide primers comprising a second barcode sequence to a barcode sequence from the substrate; thereby generating a nucleic acid signature for the particles in the partition which can be used to distinguish barcodes in separate compared to the same partition (see PCT WO2017/120531). In other aspects for deconvoluting the sequencing reads (determining whether reads come from the same or different partitions), the method can comprise: forming partitions comprising forward primers comprising a barcode and a capture sequence complementary to the 3′ sequence, or reverse complement thereof, of a target nucleic acid, wherein different partitions contain different forward primers comprising different barcode sequences, and a partition ID tag oligonucleotide comprising a reverse complement of the capture sequence and a variable partition ID tag sequence; in the partitions, hybridizing at least one forward primer to the partition ID tag oligonucleotide to form a hybridized product; performing amplification on the hybridized product to form amplicons, wherein at least some amplicons are formed from a forward primer and the partition ID tag oligonucleotide; and sequencing the amplicons, wherein if different forward primers form amplicons with the same variable partition ID tag sequence, the different forward primers are considered to be from the same partition. In some of those embodiments, the forward primer and partition ID tag oligonucleotide are linked to the same substrate when delivered to the partitions; or the partition ID tag oligonucleotide has a blocked 3′ end such that a polymerase cannot extend the blocked 3′ end during amplification; or the partition ID tag oligonucleotide comprises a double-stranded variable partition ID tag sequence and one or two single-stranded 3′ ends comprising the reverse complement of the capture sequence. See, e.g., PCT/US2019/015638.

The occluded nanovials or microwells can also be used to detect protein expressed, and optionally secreted, from cells. For example, in some embodiments, one or more binding agent that specifically binds to (e.g., has affinity for) a cellular target protein can be linked to the nanovials or microwells. In some embodiments, different (e.g., 2, 3, 4, or more) binding agents are present for different target proteins. Cells can subsequently be loaded into the nanovials or microwells as described above, and the openings can be occluded with one or more hydrogel beads, also as described above. Proteins secreted from the cells can accumulate in the nanovial or microwell and will bind to the affinity agent, thereby substantially preventing diffusion of the target proteins from the nanovials or microwells. In some embodiments, cell lysis can be induced as described above, allowing for release of target proteins from the cell, allowing for binding of the target proteins to the affinity agent.

Target proteins bound by the affinity agents in the occluded nanovials or microwells can then be specifically detected. In some embodiments, a second binding agent, that can be directly or indirectly labeled, is introduced to the occluded nanovials or microwells, e.g., via diffusion, and the presence, absence or quantity of the target protein can be detected. This can result in a sandwich assay format for detection of the target proteins. In some embodiments, the signal from the bound second binding agent is sufficient to allow for sorting (e.g., FACS sorting) of the nanovials, allowing for detection of the target protein(s). In some embodiments, with or without sorting, one can wash excess unbound secondary binding agent from the nanovials or microwells, followed by specific detection of signal from label on the second binding agent. As noted herein, the first, second or both binding agents are in some embodiments, antibodies or other binding molecules such as streptavidin or biotin. In some embodiments, the label is fluorescent. In some embodiments, there are multiple (e.gh., 2, 3, 4, or more) different second binding agents, each with a separate distinguishable label, allowing for separate detection (and optional sorting) of different target proteins.

Also provided are compositions as described herein or kits for performing the described methods. Exemplary compositions can include, for example a plurality of hollow nanovials (or microwells) containing one or more intact or lysed cell and one or more hydrogel bead occluding the opening in the nanovial (or microwell).

### Prophetic Example

Nanovials, which are 35 microns in diameter across the opening and also possess a patch of integrins at the base of the interior cavity are suspended in PBS, 0.1% BSA, 0.1% F68 pluronic and 15% PEG 6000. The nanovials are allowed to settle in a 2D plane at the bottom of a petri dish by gravity. Due to the center of gravity positioned away from the opening, the nanovials settle with the opening upwards. B-cells also suspended in PBS, 0.1% BSA, 0.1% F68 pluronic, and 15% PEG 6000 are allowed to settle into the nanovials through gravity. Once they fall to the basement of the openings, the cells bind to the nanovial via attaching to the integrins. Cells are loaded at lambdas of <0.06 such that the majority of nanovials have either one or none cells. Thirty-two micron in diameter clonal barcode gel beads also suspended in PBS, 0.1% BSA, 0.1% F68 pluronic, and 15% PEG 6000 are added to the petri dish and allowed to settle by gravity into the interior cavity of the nanovials. There is only sufficient space for one gel bead per nanovial to be loaded and so they do not have to be loaded at lower lambdas. The end result is that >80% of nanovials will contain a single barcode gel bead. These clonal barcode gel beads then swell and seal the opening by exchanging the buffer with a PEG free solution. Lysis reagent, containing 1% NP-40 is added to the buffer. The non-ionic detergent flows to the center of the nanovial cavity where the cell is located causing the cell to lyse. The high molecular weight DNA and RNA is trapped in the space between the nanovial and gel beads as the pore size in the nanovials and the gel beads are too small to allow for diffusion of said molecules. The nanovial—gel bead complex containing the trapped RNA and DNA is washed to remove the cell lysate, which is an inhibitor for downstream molecular biology reactions. A small molecular weight reverse transcriptase together with a nucleotide containing mastermix buffer is then added to the solution. Due to the small molecular weight and thus small size of the enzymes, they and the mastermix diffuses to the trapped DNA and RNA. Once the poly A tails of mRNA bind to the polyT primer that is part of the barcode oligonucleotide, reverse transcription occurs resulting in the barcoding of the RNA from a single cell. To release the barcoded RNA, PEG is then added back into the solution at 15%. The gel bead shrinks due to the presence of the PEG, allowing for the free diffusion of the barcoded cDNA. Sample prep is completed in bulk followed by sequencing. Single cell RNA can be grouped and thus a single cell transcriptome can be reconstituted by binning the sequence reads according to the clonal barcode sequences.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of linking oligonucleotides to cell nucleic acids in hollow nanovials, the method comprising,
loading cells into openings in hollow nanovials;
inserting one or more hydrogel bead into the openings, thereby occluding the openings and preventing diffusion of the cells from the openings;
lysing the cells in the hollow nanovials;
linking one or more oligonucleotides to a nucleic acid from the lysed cells thereby barcoding the nucleic acid, wherein the oligonucleotides are present in the hollow nanovials before the loading, or are introduced into the hollow nanovials by the hydrogel beads.

2. The method of claim 1, wherein the occluding comprises inducing swelling of the one or more hydrogel bead inserted into the nanovials.

3. The method of claim 1, wherein the occluding comprises inducing swelling of the nanovials.

4. The method of claim 1, wherein a majority of the nanovials contain one or zero cells.

5. The method of claim 1, wherein the oligonucleotides are linked to the hydrogel beads and are optionally released from the hydrogel beads after the sealing.

6. The method of claim 5, wherein the oligonucleotides are linked to the hydrogel beads and are optionally released from the hydrogel beads before the linking.

7. The method of claim 5, wherein the oligonucleotides have a barcode sequence, wherein individual hydrogel beads comprise clonal copies of the oligonucleotides and wherein the barcode sequence linked to individual hydrogel beads are unique such that the barcode distinguishes the hydrogel bead from other hydrogel beads.

8. The method of claim 1, wherein the oligonucleotides have a barcode sequence, wherein individual nanovials comprise clonal copies of the oligonucleotides and wherein the barcode sequenced linked to individual nanovials are unique such that the barcode distinguishes the nanovial from other nanovials.

9. The method of claim 1, further comprising extracting the barcoded nucleic acids from the nanovials and combining barcoded nucleic acids from different nanovials into a bulk solution and performing nucleotide sequencing of the barcoded nucleic acids from the bulk solution.

10. The method of claim 1, wherein the linking comprises annealing the oligonucleotides to the nucleic acids and optionally performing template-dependent extension of the oligonucleotide using the nucleic acid as the template.

11. The method of claim 10, wherein the nucleic acids are DNA and the linking reagents comprise a DNA polymerase or ligase.

12. The method of claim 10, wherein the nucleic acids are RNA and the linking reagents comprise a reverse transcriptase.

13. The method of claim 1, wherein the linking comprises linking the oligonucleotides to the nucleic acids via a click chemistry reaction.

14. The method of claim 1, wherein the hollow nanovials comprise dextran and polyethylene glycol (PEG).

15. The method of claim 1, wherein the hydrogel beads comprise polyacrylamide, polystyrene, silica, polymethylmethacrylate, alginate, polyethylene glycol (PEG), nylon, or agarose.

16. The method of claim 1, wherein the hydrogel beads comprise polyacrylamide that is cross-linked with Bis-acrylylcystamine.

17. The method of claim 1, wherein the hydrogel beads comprise polyacrylamide formed from polymerizing acrylamide and a photocleavable cross-linker.

18. The method of claim 16, wherein the photocleavable cross-linker is ortho-nitrobenzyl (o-NB) bis-acrylate.

19. A plurality of hollow nanovials comprising occluded openings that prevents diffusion of cells and released cellular products, wherein at least some of the hollow nanovials comprise one or more cell and the openings are occluded by hydrogel beads blocking the openings.

20. A method of detecting cell proteins in hollow nanovials or microwells, the method comprising,
loading cells into openings in hollow nanovials or microwells;
inserting one or more hydrogel bead into the openings, thereby occluding the openings and preventing diffusion of the cells from the openings; and
detecting secreted cell proteins in the occluded nanovials.

21. The method of claim 20, wherein the nanovials or microwells comprise a binding agent that specifically binds to one or more secreted cell protein and the secreted cell proteins bind to the binding agent, and a second binding agent specific for the secreted cell protein is diffused into the occluded nanovials or microwells to specifically detect the secreted cell protein bound in the nanovials or microwells.
